(19) United States
(12) Patent Application Publication
(10) Pub. No.: US 2014/0066490 A1
(43) Pub. Date: Mar. 6, 2014

(54) SYSTEM AND METHOD FOR MODIFYING DEOXYRIBOZYMES

(71) Applicant: YEDA Research and Development Co. Ltd., Rehovot (IL)

(72) Inventors: Ehud Y. SHAPIRO, Nataf (IL); Maya KANAN-HANUM, Rehovot (IL); Yehonatan DOUEK, Rehovot (IL); Rivka ADAR, Carmei Yosef (IL)

(73) Assignee: YEDA Research and Development Co. Ltd., Rehovot (IL)

(21) Appl. No.: 13/966,337

(22) Filed: Aug. 14, 2013

Related U.S. Application Data
(60) Provisional application No. 61/682,790, filed on Aug. 14, 2012.

(51) Int. Cl. C12N 15/113; C12N 15/10

(52) U.S. Cl. CPC C12N 15/1136; C12N 15/1089
USPC 514/44 A; 506/16; 506/26; 506/40

(57) ABSTRACT

A system and method for programming DNAzymes to be utilized as programmable drugs, which are active only in the presence of specific input combinations and/or certain conditions.

### REFERENCE TO SEQUENCE LISTING

The Sequence Listing is filed herewith electronically as a separate ASCII file entitled “3037_ST25.txt”, created on Oct. 22, 2013, having 9000 bytes in size; the contents of which are hereby incorporated by reference as if fully set forth herein.

### FIELD OF THE INVENTION

The present invention relates to a system and method for manipulating and programming enzymes, and in particular, to such a system and method wherein Deoxyribozymes are preprogrammed whereby only upon specific conditions, they become active.

### BACKGROUND OF THE INVENTION

Drugs that become active only upon the presence of preprogrammed abnormal environmental conditions may enable selective molecular therapy by targeting abnormal cells without injuring normal cells (6-12).

Catalytic DNA molecules (also referred to herein as “Deoxyribozymes” or “DNAzymes”) were used as inhibitory agents in a variety of experimental disease settings, such as cancer (1-3), viral infections (4) and even HIV (5), suggesting their possible clinical utility.

### SUMMARY OF THE INVENTION

There is an unmet need for, and it would be highly useful to have, a system and/or a method for programmable DNAzymes that may be utilized as programmable apoptosis-inducing drugs, which are active only in the presence of specific input combinations and/or certain conditions.

In some demonstrative embodiments of the present invention, there is provided a system and method for preprogramming a DNAzyme into a library of Boolean logic gates selected from the group consisting of: YES, AND, NOT, OR, NAND, ANDNOT, XOR, NOR and/or 3-input-AND wherein the activity of the DNAzyme may be influenced by the presence of at least one input molecule according to the Boolean logic gates.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computer” on a “computer network”, it should be noted that optionally any device featuring a data processor and the ability to execute one or more instructions may be described as a computer, including but not limited to any type of personal computer (PC), a server, a cellular telephone, an IP telephone, a smart phone, a PDA (personal digital assistant), or a pager. Any two or more of such devices in communication with each other may optionally comprise a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1(a)-1(c) and 2(a)-2(g) are schematic illustrations of a variety of Boolean logic gates, including YES, AND, NOT, OR, NAND, ANDNOT, XOR, NOR and 3-input-AND gate, according to some demonstrative embodiments of the present invention.

FIGS. 3(a)-3(c) illustrate different uses of a library as described herein according to some demonstrative embodiments, for diagnosing and/or treating cancerous cells.

FIGS. 4(a)-4(c) illustrate the results of an ex vivo experiment and the operation of a library design within living cells, as described herein according to some demonstrative embodiments.

FIGS. 5(a)-5(d) illustrate capillary electrophoresis results for various AND gates.

### DESCRIPTION OF THE PREFERRED EMBODIMENTS

According to some demonstrative embodiments of the present invention, there is provided a system and method for modifying a DNAzyme to be catalytically active only in the existence of specific conditions.

In some embodiments, the system and method described herein may include preprogramming the DNAzyme into a library of Boolean logic gates wherein only upon specific conditions, the DNAzyme may regain its catalytic activity. According to some embodiments, the specific conditions may be determined by the presence of predefined input molecules and the Boolean logic gate rules.

In some demonstrative embodiments, the system and/or method described herein may make use of any suitable DNAzyme, including, for example, DZF DNAzyme, Dz13 DNAzyme Rs6 DNAzyme and the like. Preferably, Dz13 is the DNAzyme that may be used. Dz13 is a DNAzyme that targets c-Jun mRNA, a transcription factor found in diseased blood vessels, eyes, lungs and joints, whereas treatment using Dz13 works by destroying RNA, essentially inhibiting c-Jun expression in cells.

According to some demonstrative embodiments, the term “input molecule” or “input” may refer to any type and/or size of molecule which may affect the activity of a DNAzyme via one or more of the embodiments described herein below. The input molecule may include any DNA or RNA sequence including, for example, miRNA, mRNA and the like. Elbaz, J. et al. Nat Nanotechnol 5, 2010, incorporated by reference as if fully set forth herein, describes systems which implemented logic libraries based on DNAzymes, wherein the computational layer is dependent on the relation between inputs, and accordingly inputs depended on each other. However, the system and/or method described herein according to some demonstrative embodiments, does not require any dependence between inputs, i.e., each input can be completely arbitrary, allowing integration of any input combinations.

Mokany, E., et al., J Am Chem Soc 132, 2010, incorporated by reference as if fully set forth herein, teach that the input specificity is determined by a maximal amount of 7 nucleotides. In contrast, the system and/or method described herein in accordance with some demonstrative embodiments, is not restricted to short input sequences. According to some embodiments, the logic gate input-binding sequences described herein may be between 1-45 nucleotides long, preferably between 18-22 nucleotides long. According to some embodiments, the number of nucleotides of the logic gate input-binding sequences described herein, allows for great specificity and minimizes identification of inappropriate inputs. According to some embodiments, this may be a key feature for using cellular markers as inputs, since a cell contains a large amount of miRNA/mRNA populations, some with common sequences.

According to some embodiments, the library of Boolean logic gates (“the library”) may include one or more of the following parameters: YES, AND, NOT, OR, NAND, ANDNOT, XOR, NOR and 3-input-AND gate. The parameters may be predetermined and may use miRNAs and/or mRNAs as inputs. In some demonstrative embodiments, the library described herein may be modular, may support arbitrary inputs and/or outputs, may be cascadable, may be highly specific and/or robust.

In some embodiments, each gate may be based on the c-jun cleaving Dz13 DNAzyme and may be utilized to induce the activity of the Dz13 DNAzyme only in the presence of specific input molecule combinations, as detailed below.

According to some embodiments, the induction of the activity of the Dz13 DNAzyme only upon the existence of certain input combinations, may enable the use of the Dz13 DNAzyme as a programmable apoptosis-inducing drug.

In some demonstrative embodiments, there is provided a system which includes a plurality of multi-component units in which computations may be performed, including, for example, computations for performing the following:

(a) splitting the DNAzyme at the core catalytic region in a way that only when an appropriate input molecule exists the complete DNAzyme complex may be formed;

(b) caging the DNAzyme arms using a stem-loop structure, which can be un-caged when an appropriate input exits; and

(c) toehold exchange in which a longer hybridization may be favored, where the presence of an input molecule may change the components' conformation.

In some demonstrative embodiments, an output of the system and/or method described herein (also referred to as “the target gene”) may be programmable and may be selected by the RNA-binding arms' sequence. Since both output and inputs are RNA molecules, unlike other miRNA based systems (for example, as described by Xie, Z., et al., Nucleic Acids Res 38, 2010, incorporated by reference as if fully set forth herein), simple reactions can be composed to form cascaded (compositional) ones, where the output RNA of one gate can serve as the input for a downstream gate.

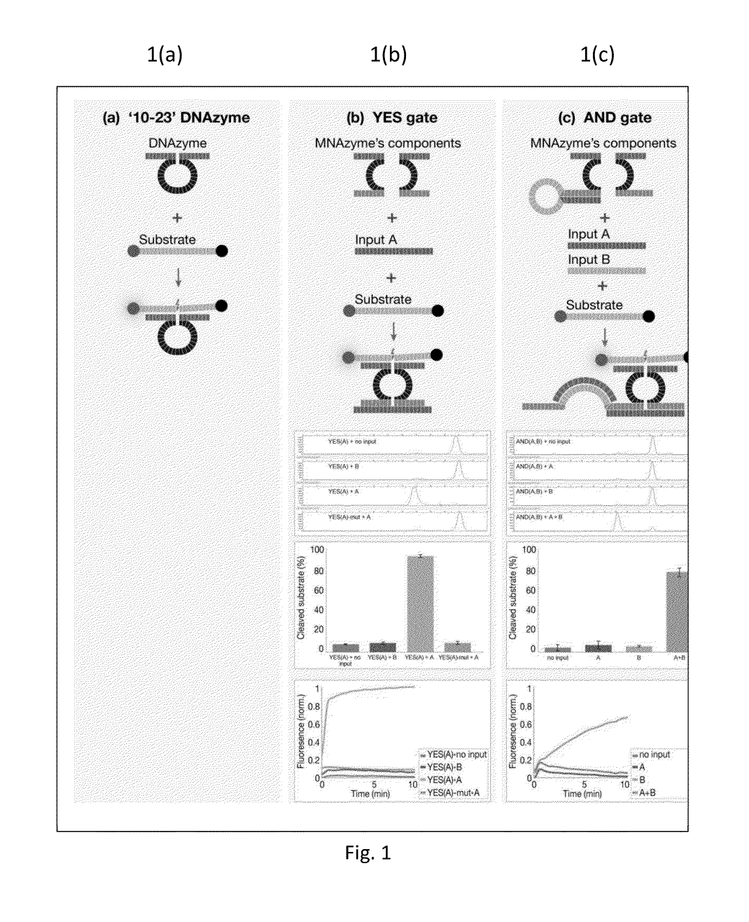

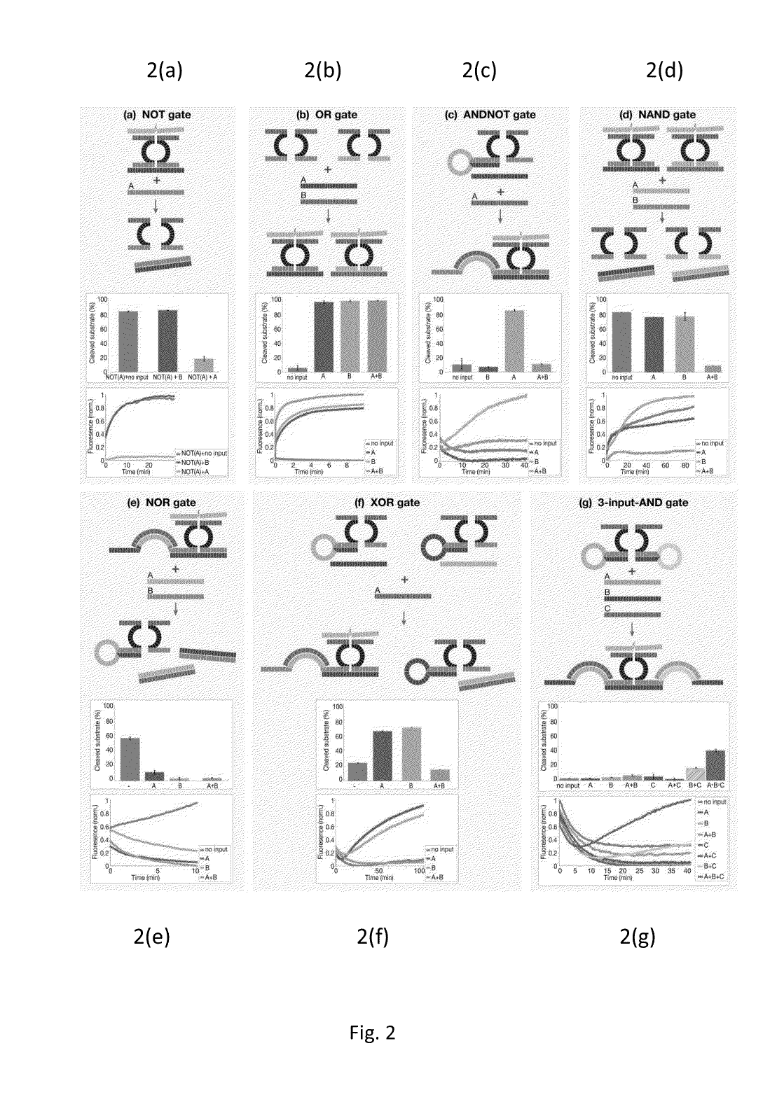

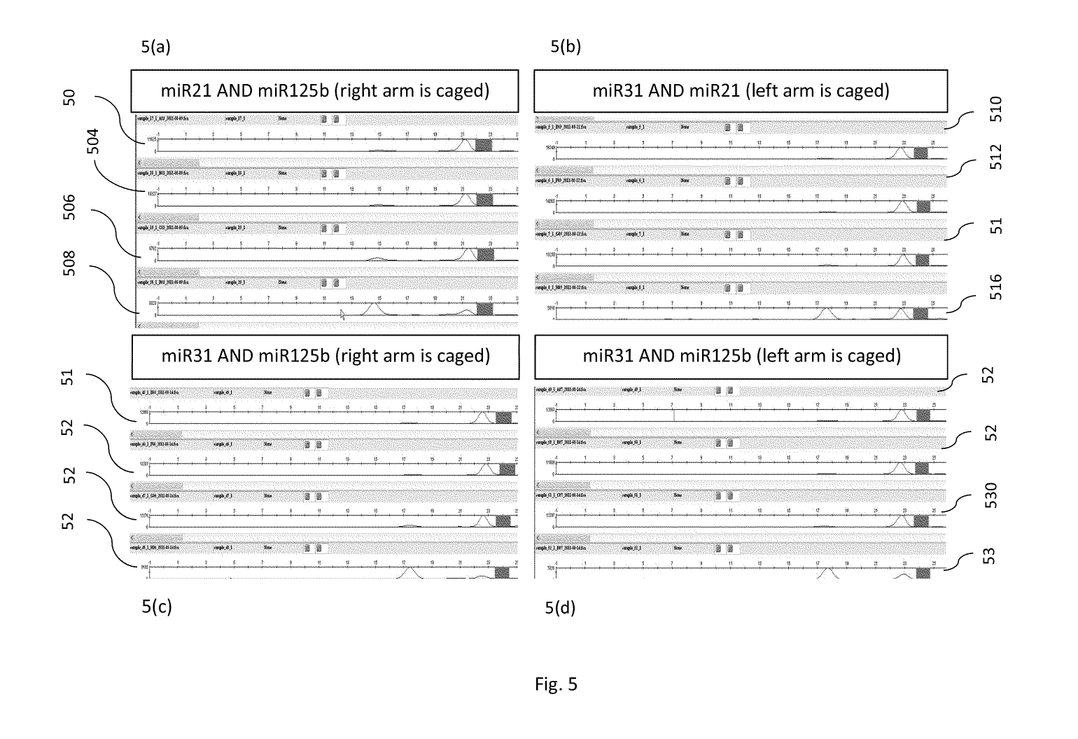

In some demonstrative embodiments of the present invention, the system and/or method described herein may support 2 or more inputs for any gate chosen, for example, for an AND gate. As described in detail below, the system and/or method described herein may include ‘locking’ input-binding arm; a ‘caging’ sequence which is complementary to the DNAzyme arm to form a stem-loop structure (FIG. 1c). When the input is present, the arm is un-caged (since the open conformation is favored) and the gate can accept the input which joins the two sub-components. 3-input-AND gate was realized by ‘locking’ both input-binding arms, as illustrated in FIG. 2g. By ‘locking’ also the substrate-binding-arms, the system can be extended to create a 5-input-AND gate. In previous designs, implementation of such a gate would require cascaded gates, each adding multiple molecular interactions. In contrast, in the system and/or method described herein, any additional input, i.e., beyond the 2 inputs, requires the addition of a single molecular interaction, keeping the total chemical complexity relatively low. Demonstration of several AND gates also shows the programmability of the gate's inputs, as illustrated for example in FIGS. 5(a)-5(d).

In contrast to previous systems described by Elbaz, J. et al. Nat Nanotechnol 5, 2010, and Macdonald, J. et al. Nano Lett 6, 2006, both incorporated by reference as if fully set forth herein, the library of gates of the system and/or method described herein may be utilized to interact with physiologic components, i.e., inputs and/or outputs.

For example, in a top-down approach, a ‘DNAzyme drug may be programmed into a ‘programmable drug’, which may operate only when certain pre-programmed conditions are met. Preferably, a member of the ‘10-23’ DNAzyme family may be utilized as hardware, over other families such as ‘E6’, ‘8-17’, that were demonstrated as the basis of Boolean logic gates and automata, as taught by Macdonald, J. et al. Nano Lett 6 2006, Stojanovic, M., et al., J Am Chem Soc 124, 2002, and Stojanovic, M. N. et al., Nat Biotechnol 21, 2003, all incoporated by reference as if fully set forth herein. A member of the ‘10-23’ DNAzyme family may be utilized as hardware mainly due to its powerful clinical antisense activity in vivo and its ability to operate in physiological conditions, including, for example, low MgCl₂ concentrations, 37° C. and the like.

In contrast to previous systems, which rely on gene transcription to perform computation, for example, as described by Xie, Z., et al., Science 333, 2011, incorporated by reference as if fully set forth herein, according to some embodiments, the system and/or method described herein possesses a computing element which may directly bind to both input and target RNA, thus, keeping chemical interactions to a minimum and reducing the number of components which should be delivered to the cell.

In some demonstrative embodiments, the system and/or method described herein includes a library of programmable DNAzymes that can operate in a cellular environment and/or in living cancer cells. The library may be modular, support arbitrary inputs and outputs, cascadable, highly specific and/or robust. Unlike single-marker diagnosis, the library of the present invention allows integration of multiple markers according to predefined rules, thus may be the basis of medical diagnosis and therapy, e.g., in the form of ‘programmable’ therapeutics.

According to some demonstrative embodiments, there is provided a method for preprogramming a set of Boolean logic gates for a nucleotide based computational machine, the method comprising providing the nucleotide based computational machine, comprising a plurality of Boolean logic gates, i.e., a DNAzyme, selected from the group consisting of: YES, AND, NOT, OR, NAND, ANDNOT, XOR, NOR and/or 3-input-AND; associating a Boolean logic gate with at least one input molecule, said input molecule influencing a behavior of said DNAzyme and wherein said DNAzyme affects a computation of said nucleotide based computational machine according to said influenced behavior by said input molecule; and providing an RNA molecule to serve as a substrate that may be cleaved by the DNAzyme, e.g., enabling a detection of an output.

According to some demonstrative embodiments, the system described herein may feature one or more user computers, for example, a plurality of user computers. Each user computer may communicate through a network, which may optionally be the Internet or any other type of computer network, with a server or a plurality of servers in any suitable configuration (for example and without limitation, as a server farm or any other distributed computing system). The user computer may communicate with the server for preprogramming a set of Boolean logic gates for a nucleotide based computational machine, as described herein in accordance with some demonstrative embodiments.

To facilitate communication between the user computers and the server and/or to facilitate the preprogramming a set of Boolean logic gates, the user computer preferably features a user interface. The user interface may optionally be implemented as a plug-in or other additional software to an existing software being operated by the user computer, such as a web browser as a non-limiting example.

Forming of the Gates

For clarity purposes the following formation of the gates has been explained and demonstrated with relation to the Dz13 DNAzyme. However, it is to be understood that any DNAzyme may be used according to the embodiments described herein.

According to some embodiments of the present invention, the library may include at least one YES gate. In some embodiments, the YES gate represents a parameter according to which a Dz13 DNAzyme becomes active, for example, when a single input molecule is present.

In some embodiments, to construct a simple YES gate, which may be active only when a single input molecule is present, the ‘10-23’ DNAzyme Dz13 (FIG. 1a) may be used. By implementing the design described by Mokany, E. et al. (J Am Chem Soc 132, 2010, incorporated by reference as if fully set forth herein), the DNAzyme's catalytic core was split into two parts between T₈ and A₉. According to these embodiments, only upon the presence of an appropriate input molecule, such as miR155, the two parts may be joined, accordingly, the DNAzyme complex may be formed and the resulting cleavage of RNA occurs (referred to herein as the ‘True’ output) (FIG. 1b).

According to some embodiments of the present invention, the library may include at least one AND gate. In some embodiments, the AND gate represents a parameter according to which a Dz13 DNAzyme becomes active, for example, when a two input molecules are present, for example, a first input molecule and a second input molecule.

In some embodiments, in order to form the AND gate, an additional binding loop, e.g., consisting of a complementary sequence for the second input, may be added and optionally followed by a ‘caging’ sequence which may be complementary to the DNAzyme arm, and accordingly forming, for example, a stem-loop structure (FIG. 1c). According to some embodiments, when the AND gate is formed, the presence of either the first input molecule or the second input molecule will not be sufficient to cause the formation of the complete DNAzyme complex. According to these embodiments, only in the presence of both input molecules, i.e., the first and second input molecules, does the formation of the complete DNAzyme complex occur.

For example, since the DNAzyme arm may have several conformations, preferably, an open conformation, when the second input molecule is present, the DNAzyme arm may be in an un-caged conformation, and the gate can accept the first input molecule which may join, for example, the two sub-components. Only when both inputs are present, the complete DNAzyme complex may be formed. According to some embodiments, caging may be done on either one of the arms, as demonstrated, for example, in FIGS. 5(a)-5(d).

According to some embodiments of the present invention, the library may include at least one NOT gate. In some embodiments, the NOT gate represents a parameter according to which a DNAzyme stops being active. According to some embodiments, the NOT gate may be formed using an additional strand which may contain the input molecule's complementary sequence (also referred to herein as ‘anti-input’ molecule). According to these embodiments, when the input molecule is present, the input molecule may ‘cancel’ the anti-input molecule, the DNAzyme's components may be separated, resulting in a ‘False’ output (FIG. 2a). According to some embodiments, the input molecule may ‘cancel’ the anti-input molecule since the input molecule and the anti-input molecules comprise a complementary sequence which allows them to hybridize to each other, thus preventing the anti-input molecule to join the two parts of the DNAzyme.

According to some embodiments of the present invention, the library may include at least one OR gate. In some embodiments, the OR gate represents a parameter according to which a Dz13 DNAzyme becomes active in the presence of at least one input molecule, for example, in the presence of either a first input molecule or in the presence of a second input molecule. For example, the OR gate may be implemented by using two YES gates operating in parallel on two different input molecules (FIG. 2b).

According to some embodiments of the present invention, the library may include parameter(s) which combine more than a single input, e.g., two inputs. In some embodiments of the present invention, the library may include at least one ANDNOT gate. In some embodiments, the ANDNOT gate represents a parameter according to which a Dz13 DNAzyme becomes active, for example, when a first input molecule is present and a second input molecule is not present. According to these embodiments, every other combination results in that the Dz13 DNAzyme remains inactive.

For example, the ANDNOT gate may be implemented by combining the AND and NOT gates' design, e.g., as described hereinabove. When both input molecules are present, the second input may cancel the anti-input molecule, resulting in a ‘False’ output (FIG. 2c).

In some embodiments of the present invention, the library may include at least one NAND gate. In some embodiments, the NAND gate represents a parameter according to which a Dz13 DNAzyme stops being active, for example, when two input molecules are present.

For example, the NAND gate may be formed by combining two NOT gates for two different input molecules. According to these embodiments, only when both input molecules are present, the two gates become inactive (FIG. 2d).

In some embodiments of the present invention, the library may include at least one NOR gate. In some embodiments, the NOR gate represents a parameter according to which the complete DNAzyme complex becomes disassembled in the presence of either input molecule out of two or more input molecules, for example, in the presence of a first input molecule or in the presence of a second input molecule.

For example, the NOR gate may be an inverse OR gate. When either the first input molecule or the second input molecule is present, the complete DNAzyme complex may become disassembled, resulting in a ‘False’ output (FIG. 2e).

In some embodiments of the present invention, the library may include at least one XOR gate. In some embodiments, the XOR gate represents a parameter according to which the DNAzyme becomes active in the presence of a first or second input molecule but is inactive in the presence of both the first and second input molecules (or none).

For example, the XOR gate may be formed by combining two ANDNOT gates. According to these embodiments, when either input molecule is present, e.g., the first input molecule or the second input molecule, it may activate one of sub-gates, but may also cancel the orthogonal sub-gate by binding to its anti-input molecule. Only when a single input molecule, but not both, is present, the gate may be active (FIG. 2(f)). According to some embodiments, the ability to activate the DNAzyme only upon the existence of specific conditions, may be used for certain diagnostic and/or medical systems, wherein only cells that meet a specific abnormal profile might be targeted, sparing healthy cells.

In some embodiments of the present invention, the library may include at least one 3-input-AND gate. In some embodiments, the 3-input-AND gate represents a parameter according to which the DNAzyme becomes active in the presence of three input molecules.

For example, the 3-input-AND gate may be formed using an AND gate in which both arms are caged. Only when all 3 input molecules are present, the complete DNAzyme complex may be formed resulting in a ‘True’ output (FIG. 2g). As previously described, overcoming false positive results can be done by utilizing both the drug and the drug-suppressor molecule (Benenson, Y et al., Nature 429, 2004, incorporated by reference as if fully set forth herein).

According to some demonstrative embodiments, the system and/or method disclosed herein may be used for diagnosing abnormal cells by using a complex Boolean expression in which input molecules, for example, any molecule for which aptamers exist, such as mRNA and miRNA may serve as ‘disease markers’. (Gil B, Kahan-Hanum M, Skirtenko N, Adar R, Shapiro E. Detection of multiple disease indicators by autonomous biomolecular computer. Nano Letters. 2011)

Reference is now made to FIGS. 1(a) to 1(c) which illustrate an in vitro YES and/or AND gates demonstration. FIG. 1(a) is an illustration of Dz13 cleaving its corresponsing fluorescently labeled RNA substrate. FIGS. 1(b) and 1(c) illustrates the operation of YES or AND gates upon presence of its inputs (upper panel); Capillary-electrophoresis demonstrates cleavage or non-cleavage of the fluorescent substrate (upper middle panel); Quantitative cleavage results of 3 independent experiments (bottom-middle panel) and Plate Reader results showing reaction kinetics (bottom panel). Each gate's results match its truth table. For YES: input A=miR155, un-proper input B=miR31; For AND: input A=miR21, input B=miR125b.

Reference is now made to FIGS. 2(a) to 2(g) which illustrate a logic gate design and implementation. Each one of FIGS. 2(a) to 2(g) demonstrates the following: upper panel, schematic gate operation in the presence of one combination of its inputs; middle panel, quantitative cleavage results of its entire set of inputs within independent experiments; bottom panel, reaction kinetics. The results of each gate matches its truth table and inputs were chosen as follows: (a), for NOT: input A=miR31; (b), for OR: input A=miR21, input B=miR125b; (c), (e) and (0, for ANDNOT, NOR & XOR: input A=miR31, input B=miR125b; (d), for NAND: input A=miR31, input B=miR155; (g), for 3-input-AND gate, input A=miR31, input B=miR21 and input C=miR125b.

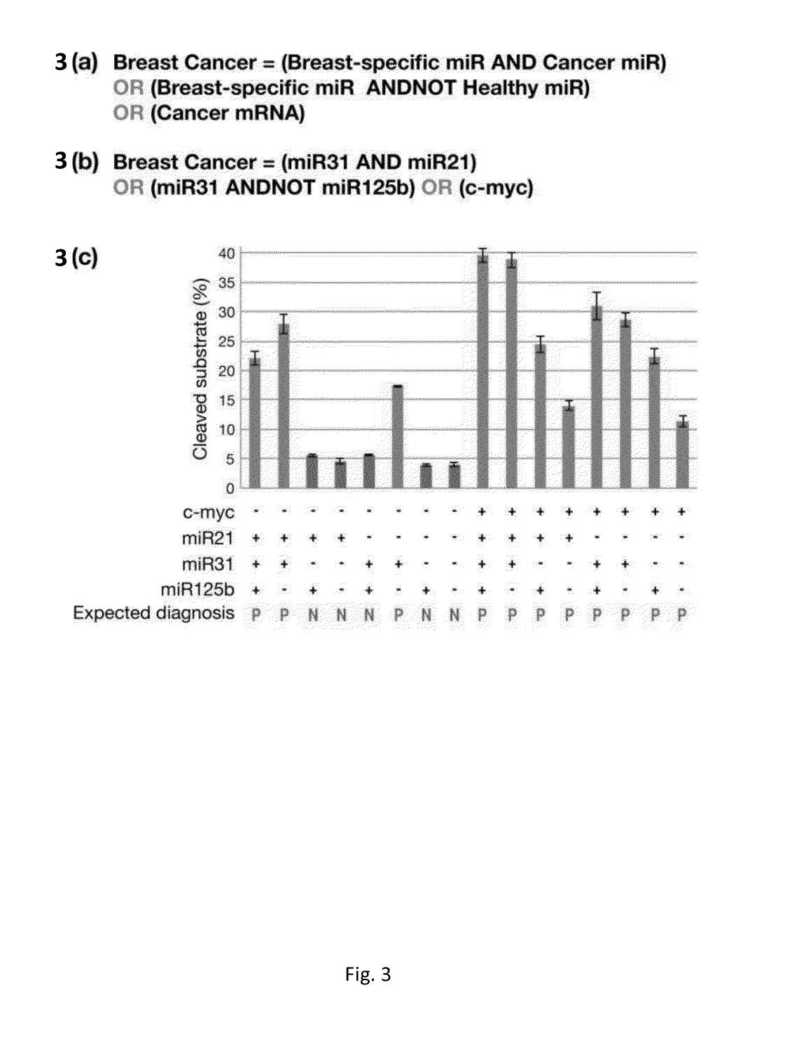

Reference is made to FIGS. 3(a)-3(c) which demonstrates an expression profile of breast cancer in cell lysates. FIGS. 3(a) and 3(b) demonstrate the definition of Boolean expressions representing positive breast cancer diagnosis. FIG. 3(c) demonstrates capillary electrophoresis results for the complex Boolean expression. According to some demonstrative embodiments, it is possible to perform experiments using the same conditions as the in vitro experiments in previous figures, by replacing DDW with cell lysate. According to some embodiments, only upon fulfillment of conditions which meet the requirements defined for ‘breast cancer’ the DNAzyme may become active and its substrate may therefore be cleaved.

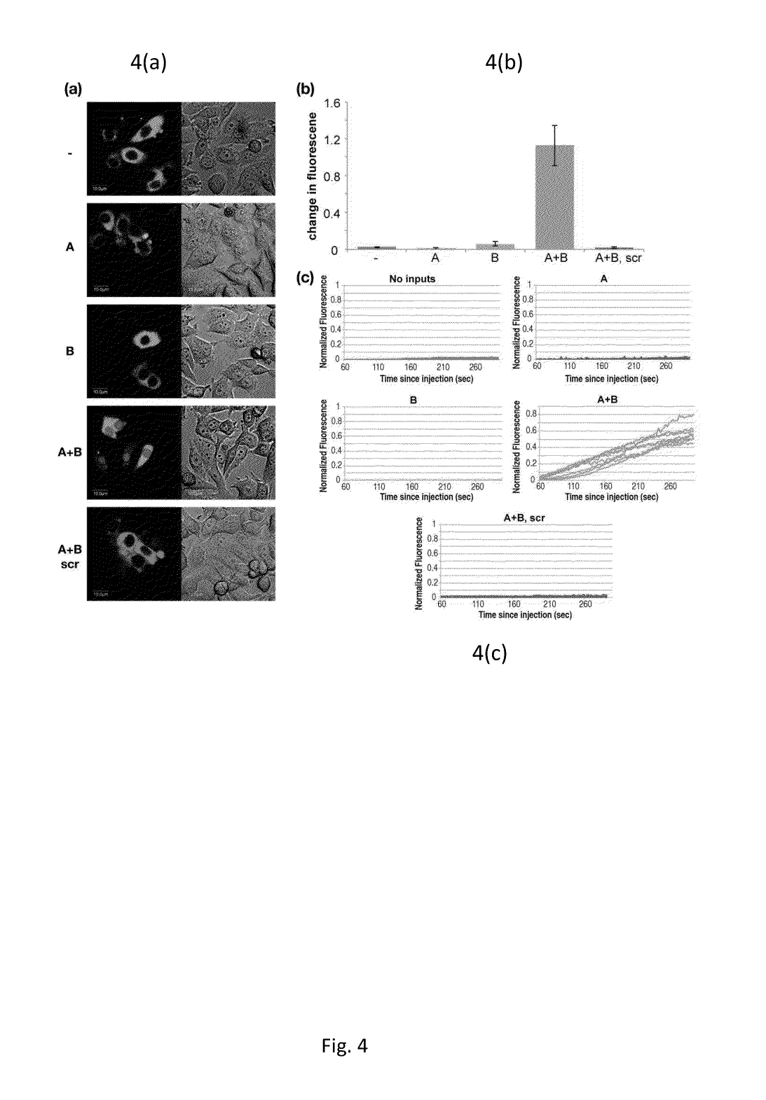

Reference is now made to FIGS. 4(a) to 4(c) which illustrate a DNAzyme-based AND gate operation within cancerous living cells. FIG. 4(a) demonstrates fluorescent view of the injected cells; FIG. 4(b) demonstrates phase view of the cells during and after injection. For each inputs combination 15 cells were microinjected with the following mixtures: (1) AND gate components; (2) combination of miRNA inputs (A=miR21, B=mir125b), (3) a fluorescent-quenched substrate (red) and (4) green Dextran (70 KDa), which was used to normalize and mark injected cells. In the experiment according to FIG. 4(a) the representative injected cells were imaged for 5 minutes after injection. Only when both inputs were present (A+B), the red substrate was cleaved and therefore visible. The term ‘scr’ represents injections of a scrambled DNAzyme's component sequence, which were otherwise identical to the normal injections; As can be seen in FIG. 4(b) average of relative change is shown in red fluorescence in 15 cells, 5 minutes after microinjection (change was calculated as: normalized fluorescence 5 minutes post injection minus normalized fluorescence 1 minute post injection). Error bars represent standard error; FIG. 4(c) demonstrates the kinetic results in living cells.

Reference is now made to FIGS. 5(a) to 5(d) which illustrate a fluorescent substrate length. In FIG. 5(a) the order of inputs is as follows: no inputs (502), A (504), B (506), A+B (508). In FIG. 5(b) the order of inputs is as follows: no inputs (510), A (512), B (514), A+B (516). In FIG. 5(c) the order of inputs is as follows: no inputs (518), A (520), B (522), A+B (524). In FIG. 5(d) the order of inputs is as follows: no inputs (526), A (528), B (530), A+B (532). All sequences can be found in Table 1.

### Example 1

### Experimental Implantation

DNAzyme Design and Synthesis

Programmable DNAzymes are based on the ‘10-23’ DNAzyme, with the exception that their catalytic core was split between T₈ and A₉ (based on previous MNAzyme design (Mokany, E. et al., J Am Chem Soc 132, 2010)) resulting in two parts, each containing a 9 nt substrate binding region and a 10-11 nt input binding region (input is split across the two parts). Prior to any experiment, gate components were heated to 99° C. (in a 50 mM NaCl solution) and slowly cooled to 10° C. to allow hybridization and stored in −20° C. Synthetic single-stranded RNA substrate was labeled by a FAM fluorophore in its 3′ end. Unmodified DNA sequences were obtained from Sigma-Aldrich® (Standard Desalted). Modified DNA sequences and RNA sequences were obtained from Integrated DNA Technologies (IDT) and were HPLC Purified. Oligos were stored in −20° C. in TE buffer. All sequences can be found in Table 1.

TABLE 1
DNAzyme library sequences:

| Component | Name | Sequence |
|---|---|---|
| | Dz13 | CGGGAGGAA GGCTAGCTACAACGA GAGGCGTTG Ti |
| | (SEQ ID | |
| | NO: 1) | |
| | Dz13 | CAA CGC CUC GUU CCU CCC G/FAM/ |
| | substrate | |
| | (SEQ ID | |
| | NO: 2) | |
| YES gate | YD157.1 | CGGGAGGAAGGCT AGCTCATCTTGCCT |
| | (SEQ ID | |
| | NO: 3) | |
| | YD157.2 | AGCTATGCCAGACAACGAGAGGCGTTG |
| | (SEQ ID | |
| | NO: 4) | |
| | YD157.1- | CGGGAGGAAGGCTACCTCATCTTGCCT |
| | mut | |
| | (SEQ ID | |
| | NO: 5) | |
| OR gate | YD291.1 | CGGGAGGAAGGCT AGCTCTGATAAGCTA |
| | (SEQ ID | |
| | NO: 6) | |
| | YD291.2 | TCAACATCAGT ACAACGAGAGGCGTTG |
| | (SEQ ID | |
| | NO: 7) | |
| | YD312.1 | CGGGAGGAAGGCTAGCTGGTCTCAGGGA |
| | (SEQ ID | |
| | NO: 8) | |
| | YD312.2 | TCACAAGTTAG ACAACGAGAGGCGTTG |
| | (SEQ ID | |
| | NO: 9) | |
| AND gate | YD291.2 | TCAACATCAGT ACAACGAGAGGCGTTG |
| | (SEQ ID | |
| | NO: 7) | |
| | YD318 | CGGGAGGAAGGCT AGCTCTGATAAGCTA |
| | (SEQ ID | TCACAAGTTAGGGTCTCAGGGA |
| | NO: 10) | TAGCTTATCAG AGCTAGCCT (Ti) |
| NOT gate | YD413.1 | CGGGAGGAAGGCT AGCTCGTGATAGG |
| | (SEQ ID | |
| | NO: 11) | |
| | YD413.2 | AATGCTAATACAACGAGAGGCGTTG |
| | (SEQ ID | |
| | NO: 12) | |
| | YD399.3 | ACCCCTATCACGATTAGCATTAA |
| | (SEQ ID | |
| | NO: 13) | |
| ANDNOT gate | YD412.2 | GGCAAGATGACAACGAGAGGCGTTG |
| | (SEQ ID | |
| | NO: 14) | |
| | YD317.3 | AGCTATGCCAGCATCTTGCCT |
| | (SEQ ID | |
| | NO: 15) | |
| | YD404 | CGGGAGGAAGGCT AGCTCTGGCATAGC |
| | (SEQ ID | TCACAAGTTAGGGTCTCAGGGA |
| | NO: 16) | GCTATGCCAG AGCTAGCCT |
| NAND gate | YD413.1 | CGGGAGGAAGGCT AGCTCGTGATAGG |
| | (SEQ ID | |
| | NO: 11) | |
| | YD413.2 | AATGCTAATACAACGAGAGGCGTTG |
| | (SEQ ID | |
| | NO: 12) | |
| | YD399.3 | ACCCCTATCACGATTAGCATTAA |
| | (SEQ ID | |
| | NO: 13) | |
| | YD414.1 | GGGAGGAAGGCT AGCTCTGGCATAG |
| | (SEQ ID | |
| | NO: 17) | |
| | YD414.2 | GGCAAGATGACAACGAGAGGCGTT |
| | (SEQ ID | |
| | NO: 18) | |
| | YD317.3 | AGCTATGCCAGCATCTTGCCT |
| | (SEQ ID | |
| | NO: 15) | |
| XOR gate | YD412.2 | GGCAAGATGACAACGAGAGGCGTTG |
| | (SEQ ID | |
| | NO: 14) | |
| | YD317.3 | AGCTATGCCAGCATCTTGCCT |
| | (SEQ ID | |
| | NO: 15) | |
| | YD404 | CGGGAGGAAGGCT AGCTCTGGCATAGC |
| | (SEQ ID | TCACAAGTTAGGGTCTCAGGGA |
| | NO: 16) | GCTATGCCAG AGCTAGCCT |
| | YD333.1 | CGGGAGGAAGGCT AGCTCTAACTT |
| | (SEQ ID | |
| | NO: 19) | |
| | YD333.2.2 | TTGT GGTCTCAGGGA ATG CCAGCATCTTGCCT |
| | (SEQ ID | TCCCTGAGACCACAACGAGAGGCGTTG |
| | NO: 20) | |
| | YD333.3 | TCACAAGTTAGGGTCTCAGGGA |
| | (SEQ ID | |
| | NO: 21) | |
| NOR gate | YD412.2 | GGCAAGATGACAACGAGAGGCGTTG |
| | (SEQ ID | |
| | NO: 14) | |
| | YD418 | CGGGAGGAAGGCT AGCTCTGGCATAGC |
| | (SEQ ID | TCCCTGAGACCCTAACTTGTGA |
| | NO: 22) | GCTATGCCAG AGCTAGCCT |
| | YD317.3 | AGCTATGCCAGCATCTTGCCT |
| | (SEQ ID | |
| | NO: 15) | |
| | YD333.3 | TCACAAGTTAGGGTCTCAGGGA |
| | (SEQ ID | |
| | NO: 21) | |
| 3-input-AND | YD365 | TCGTTGT ACTGATGTTGA AGCTATGCCAGCATCTTGCCT |
| gate | (SEQ ID | TCAACATCAGTACAACGAGAGGCGTTG |
| | NO: 23) | |
| | YD319 | CGGGAGGAAGGCT AGCTCTGATAAGCTA |
| | (SEQ ID | TCACAAGTTAGGGTCTCA |
| | NO: 24) | TAGCTTATCAG AGCTAGCC |
| Supplementary | YD312.1 | CGGGAGGAAGGCTAGCTGGTCTCAGGGA |
| AND Gate 1 | (SEQ ID | |
| (miR21 AND | NO: 8) | |
| miR125b) | | |
| | YD324 | TCGTTGTCTAACTTGTGATCAACATCAGTCTGATAAGCTA |
| | (SEQ ID | TCACAAGTTAG ACAACGAGAGGCGTTG |
| | NO: 25) | |
| Supplementary | YD344 | CGGGAGGAAGGCT AGCTCTGATAAGCTA |
| AND Gate 2 | (SEQ ID | AGCTATGCCAGCATCTTGCCT |
| (miR31 AND | NO: 26) | TAGCTTATCAG AGCTAGCCT |
| miR21) | | |
| | YD291.2 | TCAACATCAGT ACAACGAGAGGCGTTG |
| | (SEQ ID | |
| | NO: 7) | |
| Supplementary | YD343 | TCGTTGTCTAACTTGTGAATGCCAGCATCTTGCCT |
| AND Gate 3 | (SEQ ID | TCACAAGTTAG ACAACGAGAGGCGTTG |
| (miR31 AND | NO: 27) | |
| miR125b) | | |
| | YD312.1 | CGGGAGGAAGGCTAGCTGGTCTCAGGGA |
| | (SEQ ID | |
| | NO: 8) | |
| Supplementary | YD360 | CGGGAGGAAGGCTAGCTGGTCTCAGGGA |
| AND Gate 4 | (SEQ ID | AGCTATGCCAGCATCTT |
| (miR31 AND | NO: 28) | TCCCTGAGACC AGCTAGCC |
| miR125b) | | |
| | YD312.2 | TCACAAGTTAG ACAACGAGAGGCGTTG |
| | (SEQ ID | |
| | NO: 9) | |
| Inputs | Mir155 | UUAAUGCUAAUCGUGAUAGGGGU |
| | (SEQ ID | |
| | NO: 29) | |
| | Mir31 | AGGCAAGAUGCUGGCAUAGCU |
| | (SEQ ID | |
| | NO: 30) | |
| | Mir125b | UCCCUGAGACCCUAACUUGUGA |
| | (SEQ ID | |
| | NO: 31) | |
| | Mir21 | UAGCUUAUCAGACUGAUGUUGA |
| | (SEQ ID | |
| | NO: 32) | |

Ti denotes an inverted thymidine.

In Vitro Cleavage Experiments

Experiments were performed by preparing a reaction mixture containing the DNAzyme's fluorogenic RNA substrate (0.1 μM final concentration) in 10 μl reaction buffer (Tris-HCl 50 mM, pH 7.5, 150 mM NaCl, 10 mM MgCl2) and 1 μl final concentration of each input (for the NAND & XOR gates, 2 μM or 10 μM final concentration was used, respectively). Computation was initiated by the addition of 1 μl of each of the DNAzyme components (2 μM final concentration) followed by further incubation for 20 minutes (unless stated otherwise) at 37° C. The reaction was terminated by transferring 0.5 μl of each sample to 22 μl Formamide containing GeneScan LIZ120 size standards (diluted 1:40 in Formamide) (Applied Biosystems). Samples were run on a capillary electrophoresis machine (ABI Prism, Avant-3100, Applied Biosystems) and analyzed using the GeneMapper software.

In Vitro Kinetic Experiments

Experiments were performed by preparing a reaction mixture that contained the DNAzymes quenched/fluorogenic RNA substrate. The quencher used was 3BHQ (3′ end) and it corresponding fluorophore was i6-FAMK (positioned internally between C4 and G5) Sequence: CAAC/i6-FAMK/GCCTCguTCCTCCCG/3BHQ_{—}1/(SEQ ID NO:33), lowercase letters designate RNA nucleotides). Kinetics measurements were done using Tecan Infinite® 200 microplate-reader. Fluorescence based experiments were done in a total volume of 30 μl in the same conditions as above, in 10 fold concentrations (1 μM substrate, 20 μM inputs and DNAzyme components). Excitation and emission values for the FAM fluorophore were set to 485 nm and 518 nm, respectively. Initial fluorescence was measured for 3 seconds prior to reaction initialization by addition of MgCl₂, to establish the fluorescence baseline.

Lysate Preparation

For experiments conducted in cell lysates, MCF7 cells grown in 10 cm dishes (70% confluency) were trypsinized, pelleted and washed twice in cold PBS, followed by PBS was replacement by double distilled water (DDW) as a hypotonic solution to initiate cell lysis for 15 minutes (on ice). Finally, cells were passed 15 times through a 3 μm needle resulting in cell disruption and mechanic lysis. Lysates were stored in −80° C. Lysate experiments were performed using the same conditions as for the in vitro experiments mentioned above, by replacing DDW with cell lysate (0.37 mg/ml total protein concentration, as measured by a BCA protein assay). miR21, miR31, miR125b and myc concentrations were 2 μM, 4 μM, 10 μM and 184 ng/μl respectively. Computation was initiated by the addition of the DNAzyme's fluorogenic RNA substrate (0.3 μM final concentration) followed by further incubation for 30 minutes.

In Vitro Transcription

c-myc mRNA was transcribed in-vitro using Ambion's T7 MegaScript kit, according to the manufacturer's instructions. For template preparation, the myc gene was amplified from the pBS-human-cmyc plasmid by PCR with the following primers:

(SEQ ID NO: 34)

| | |
|---|---|
| Fwd: | TAATACGACTCACTATAGTTCGGGTAGTGGAAAACCAG; |

(SEQ ID NO: 35)

| | |
|---|---|
| Rev: | TTTCCGCAACAAGTCCTCTT. |

Cell Culture

Human MCF7 breast carcinoma cells were obtained from American Type Culture Collection (ATCC) and cultured in RPMI (Gibco-BRL) pH 7.4, supplemented with 10% fetal calf serum (FCS). All media were supplemented with 10 mg/ml streptomycin and 10 IU/ml penicillin All cultures were incubated in a humidifed atmosphere of 5% CO2 at 37° C. Cells were passaged routinely by trypsinization. Subconfluent MCF7 cells (70±90%) were incubated overnight prior to microinjections.

Microinjections and Live Computation Detection

The biomolecular devices were delivered into human living MCF7 cells, by live microinjections (Appendorf micro-injector, InjectMan-NI2) using the Confocal microscope (Olympus Confocal system, Flowview 500). Live detection of displacement, hybridization and cleavage was accomplished by using quenched/fluorescent labeled substrate. The quencher used was 3BHQ (3′ end) and its corresponding fluorophore was i6-Cy3 (positioned internally between C4 and G5). Sequence: CAAC/i6-Cy3/GCCTCguTCCTCCCG/3BHQ_{—}1/(SEQ ID NO:36) lowercase letters designate RNA nucleotides. Cells were imaged with an IX70-based Olympus Confocal laser-scanning microscope using ×20 (UplanApo) objective, in a sequential mode. ex vivo assays were performed by microinjection of the substrate in a concentration of 1 μM and RNA inputs, and logic gates molecules, in a concentration of 20 μM. Cells were co-injected with Dextran (Oregon) to mark injected cells and to quantify injection efficiency. Fluorescent changes were normalized using dextran. To protect logic gate components, DNAzymes containing an additional 3′ inverted thymidine were used. Analysis was performed using the FlowView software.

### Example 2

In order to demonstrate the ability to diagnose unhealthy cells by using parameters/gates as defined in the library described according to the present invention, it is possible to use the example of breast cancer. However, it is understood that many other diseases are also relevant, for example, any disease that includes abnormal expression of one or more miRNA, mRNA or any input molecule for which aptamers exist. For example miR-99a and miR197 are under-expressed in psoriatic skin, whereas miR-146a is over-expressed in T cells from patients with rheumatoid arthritis, and may thus be used as input molecules according to some demonstrative embodiments described herein.

According to some embodiments of the present invention, with relation to a system and/or method for the diagnosis and/or treatment of breast cancer, the following expression may be chosen for the definition of the Boolean gates: Breast Cancer=(Breast-specific miRNA AND Cancer miRNA) OR (Breast-specific miRNA ANDNOT Healthy miRNA) OR (Cancer mRNA).

A breast cell is considered ‘cancerous’ if it either possesses:
(1) combinations of breast-specific microRNA and a ‘cancerous’ microRNA or
(2) breast-specific miRNA but lacks the ‘health indicative’ miRNA or
(3) cancerous mRNA. The inputs demonstrated are miR31 as a breast-specific miRNA, miR21 as a strong markers of breast cancer and miR125b as health indicative miRNA.

According to some embodiments, a c-myc oncogene may serve as a cancerous mRNA marker. Therefore, according to some embodiments, the expression can be further described as:

Breast Cancer=(miR31 AND miR21) OR (miR31 ANDNOT miR125b) OR (c-myc).

### Example 3

### Applicability in a Cellular Environment

In some demonstrative embodiments, to indicate the ability of the system and/or method described herein to be applicable in both diagnosing and/or treating cancerous cells the above expression may be solved in a cellular environment e.g., cancer cells lysate.

According to this example, the gates may be based on Dz13 DNAzyme. Accordingly, a ‘true’ output represents an active state of the DNAzyme, which can potentially cleave the c-jun mRNA. In some embodiments, cleaving the c-jun mRNA may be a key step of an apoptosis-based ‘treatment’.

### Example 4

### Applicability in Human Cancer Cells

In this example, beacon-like substrate which contains a fluorophore and a quencher is used. Only when the substrate is cleaved by a DNAzyme, the fluorophore and quencher are separated resulting in light emission. The library's components, along with the substrate and input were introduced into cells by microinjection using a Confocal microscope. Microinjection, although sometimes harmful to living cells, provides the best control over time, enabling demonstration of computation in its first several minutes. To protect the libraries' components from nucleases, an inverted thymidine modification is added to the 3′ end. As shown in FIG. 4, the AND gate, representing the library's design, operates within living cells, ex vivo, and computations are very rapid (less than 5 minutes).

### Example 5

### Various AND Gates

To demonstrate that the exemplary designs described herein are not sequence-dependent and can accept various inputs, various AND gates were created. These also demonstrates that caging can be done on either one of the AND gate components (right or left arm). FIG. 5 shows capillary electrophoresis results for various AND gates; the experiments were performed as previously described. Briefly, FIG. 5A shows results for miR21 AND miR125b (right arm is caged); FIG. 5B shows results for miR31 AND miR21 (left arm is caged); FIG. 5C shows results for miR31 AND miR125b (right arm is caged); and FIG. 5D shows results for miR31 AND miR125b (left arm is caged). As previously described, each panel shows the substrate length, confirming that different sequences can be successfully used in implementing the above described designs according to some embodiments of the present invention.

### REFERENCES

1 Dass, C. R., Galloway, S. J. & Choong, P. F. Dz13, a c-jun DNAzyme, is a potent inducer of caspase-2 activation. Oligonucleotides 20, 137-146, doi:10.1089/oli.2009.0226 (2010).
2 Wu, Y. et al. Inhibition of bcr-abl oncogene expression by novel deoxyribozymes (DNAzymes). Hum Gene Ther 10, 2847-2857, doi:10.1089/10430349950016573 (1999).
3 Dass, C. R., Choong, P. F. & Khachigian, L. M. DNAzyme technology and cancer therapy: cleave and let die. Mol Cancer Ther 7, 243-251, doi:7/2/243 [pil] 10.1158/1535-7163.MCT-07-0510 (2008).
4 Reyes-Gutierrez, P. & Alvarez-Salas, L. M. Cleavage of HPV-16 E6/E7 mRNA mediated by modified 10-23 deoxyribozymes. Oligonucleotides 19, 233-242, doi:10.1089/oli.2009.0193 (2009).
5 Jakobsen, M. R., Haasnoot, J., Wengel, J., Berkhout, B. & Kjems, J. Efficient inhibition of HIV-1 expression by LNA modified antisense oligonucleotides and DNAzymes targeted to functionally selected binding sites. Retrovirology 4, 29, doi:1742-4690-4-29 [pil] 10.1186/1742-4690-4-29 (2007).
6 Xie, Z., Liu, S., Bleris, L. & Benenson, Y. Logic integration of mRNA signals by an RNAi-based molecular computer. Nucleic Acids Res 38, 2692-2701, doi:gkq117 [pil] 10.1093/nar/gkq117 (2010).
7 Chen, Y. Y., Jensen, M. C. & Smolke, C. D. Genetic control of mammalian T-cell proliferation with synthetic RNA regulatory systems. Proc Natl Acad Sci USA 107, 8531-8536, doi:1001721107 [pii] 10.1073/pnas.1001721107 (2010).
8 Culler, S. J., Hoff, K. G. & Smolke, C. D. Reprogramming cellular behavior with RNA controllers responsive to endogenous proteins. Science 330, 1251-1255, doi:330/6008/1251 [pii] 10.1126/science.1192128 (2010).
9 Benenson, Y., Gil, B., Ben-Dor, U., Adar, R. & Shapiro, E. An autonomous molecular computer for logical control of gene expression. Nature 429, 423-429 (2004).
10 Elbaz, J., Shimron, S. & Willner, I. pH-triggered switchable Mg2+-dependent DNAzymes. Chem Commun (Camb) 46, 1209-1211, doi:10.1039/b919417g (2010).
11 Nissim, L. & Bar-Ziv, R. H. A tunable dual-promoter integrator for targeting of cancer cells. Mol Syst Biol 6, 444, doi:msb201099 [pil] 10.1038/msb.2010.99 (2010).
12 Xie, Z., Wroblewska, L., Prochazka, L., Weiss, R. & Benenson, Y. Multi-input RNAi-based logic circuit for identification of specific cancer cells. Science 333, 1307-1311, doi:333/6047/1307 [pii] 10.1126/science.1205527 (2011).
13 Dass, C. R. & Choong, P. F. C-jun: pharmaceutical target for DNAzyme therapy of multiple pathologies. Pharmazie 63, 411-414 (2008).
14 Mokany, E., Bone, S. M., Young, P. E., Doan, T. B. & Todd, A. V. MNAzymes, a versatile new class of nucleic acid enzymes that can function as biosensors and molecular switches. J Am Chem Soc 132, 1051-1059, doi:10.1021/ja9076777 (2010).
15 Liang, Y., Ridzon, D., Wong, L. & Chen, C. Characterization of microRNA expression profiles in normal human tissues. BMC Genomics 8, 166, doi:1471-2164-8-166 [pil] 10.1186/1471-2164-8-166 (2007).
16 Heneghan, H., Miller, N., Lowery, A., Sweeney, K. & Kerin, M. MicroRNAs as Novel Biomarkers for Breast Cancer. J Oncol 950201 (2009).
17 Meyer, N. & Penn, L. Z. Reflecting on 25 years with MYC. Nat Rev Cancer 8, 976-990, doi:nrc2231 [pil] 10.1038/nrc2231 (2008).
18 Elbaz, J. et al. DNA computing circuits using libraries of DNAzyme subunits. Nat Nanotechnol 5, 417-422, doi:nnano.2010.88 [pil] 10.1038/nnano.2010.88 (2010).
19 Macdonald, J. et al. Medium scale integration of molecular logic gates in an automaton. Nano Lett 6, 2598-2603 (2006).
20 Stojanovic, M., Mitchell, T. & Stefanovic, D. Deoxyribozyme-based logic gates. J Am Chem Soc 124, 3555-3561, doi:ja016756v [pii] (2002).
21 Stojanovic, M. N. & Stefanovic, D. A deoxyribozyme-based molecular automaton. Nat Biotechnol 21, 1069-1074, doi:nbt862 [pil] 10.1038/nbt862 (2003).
22 Zhang, L. et al. Angiogenic inhibition mediated by a DNAzyme that targets vascular endothelial growth factor receptor 2. Cancer Res 62, 5463-5469 (2002).
23 Fahmy, R. G. et al. Suppression of vascular permeability and inflammation by targeting of the transcription factor c-Jun. Nat Biotechnol 24, 856-863, doi:nbt1225 [pil] 10.1038/nbt1225 (2006).
24 Santoro, S. W. & Joyce, G. F. Mechanism and utility of an RNA-cleaving DNA enzyme. Biochemistry 37, 13330-13342, doi:bi9812221 [pil] 10.1021/bi9812221 (1998).

While the invention has been described with respect to a limited number of embodiments, it will be appreciated that many variations, modifications and other applications of the invention may be made.

What is claimed is:

1. A method for preprogramming a DNAzyme into a library of Boolean logic gates selected from the group consisting of: YES, AND, NOT, OR, NAND, ANDNOT, XOR, NOR and/or 3-input-AND;
wherein an activity of said DNAzyme is influenced by the presence of at least one input molecule according to said Boolean logic gates.

2. The method of claim 1, wherein said activity includes said DNAzyme becoming active and cleaving an RNA substrate.

3. The method of claim 2, wherein said substrate includes c-jun mRNA.

4. The method of claim 1, wherein said activity includes said DNAzyme becoming inactive.

5. The method of claim 1, wherein said at least one input molecule includes a DNA or RNA sequence.

6. The method of claim 5, wherein said at least one input molecule includes an miRNA and/or mRNA sequence.

7. The method of claim 1, wherein the DNAzyme is a Dz13 DNAzyme.

8. The method of claim 1, wherein said preprogramming includes:
a. Splitting the DNAzyme at the core catalytic region in a way that only when an appropriate input molecule exists the complete DNAzyme complex may be formed;
b. Caging the DNAzyme arms using a stem-loop structure, which can be un-caged when an appropriate input exits.
c. performing a toehold exchange in which a longer hybridization may be favored, where the presence of an input molecule may change the components' conformation.

9. A method for preprogramming a set of DNAzyme Boolean logic gates for a displacement based computational machine, comprising
defining a plurality of Boolean logic gates selected from the group consisting of: YES, AND, NOT, OR, NAND, ANDNOT, XOR, NOR and/or 3-input-AND; and
associating a Boolean logic gate with at least one input molecule, said input molecule influencing a behavior of said DNAzyme and wherein said DNAzyme affects a computation of said nucleotide based computational machine according to said influenced behavior by said at least one input molecule.

10. A method of treating cancer in a patient in need thereof, comprising inducing apoptosis in cancer cells of said patient by administering a programmable apoptosis-inducing drug comprising:
a Dz13 DNAzyme preprogrammed into a library of Boolean logic gates selected from the group consisting of: YES, AND, NOT, OR, NAND, ANDNOT, XOR, NOR and/or 3-input-AND;
activating said Dz13 DNAzyme in the presence of at least one input molecule according to said Boolean logic gates; and
wherein said at least one input molecule is present/absent only in said cancer cells.

11. A system for programming a DNAzyme into a library of Boolean logic gates, the system comprising:
a. a device comprising a user interface for defining said Boolean logic gates; and
b. a display for displaying results of said definition.

12. A programmable DNAzyme library comprising a plurality of DNAzymes, said DNAzymes accepting miRNA and mRNA as input molecules, adapted to induce apoptosis in a cell only when exposed to a specific input molecule.

13. The library of claim 13, wherein each DNAzyme is based upon a Dz13 DNAzyme.

14. The library of claim 13, comprising a YES gate DNAzyme active upon receiving a single specific input molecule, said YES gate DNAzyme comprising a catalytic core, said core being split into two parts such that said two parts of said core join only upon receiving an appropriate input molecule, thereby forming said DNAzyme and cleaving a target sequence.

15. The library of claim 14, comprising an AND gate DNAzyme, said AND gate DNAzyme activating upon receiving a plurality of input molecules, said AND gate DNAzyme comprising said YES gate DNAzyme, two binding loop sequences for said two input molecules and a caging sequence to control access to at least one of said binding loop sequences, wherein said binding loop sequences and said caging sequence are connected to said YES gate DNAzyme in such a manner that upon receiving said plurality of input molecules, said caging sequence enables access to said at least one of said binding loop sequences, and said AND gate DNAzyme is activated and cleaves said target sequence.

16. The library of claim 15, comprising a multi input AND gate DNAzyme, wherein said multi input AND gate DNAzyme accepts at least three inputs to become activated and cleave said target sequence.

17. The library of claim 15, comprising a NOR gate DNAzyme, said NOR gate DNAzyme comprising an inverse AND gate DNAzyme, such that if either input molecule is present, said NOR gate DNAzyme is not active.

18. The library of claim 14, comprising a NOT gate DNAzyme, said NOT gate DNAzyme activating upon receiving a first input molecule but deactivating upon receiving a second input molecule, said NOT gate DNAzyme comprising said YES gate DNAzyme, two binding loop sequences for said two input molecules, wherein said binding loop sequences are connected to said YES gate DNAzyme in such a manner that upon receiving said second input molecule, said binding loop sequence for said second input molecule blocks access to said binding loop sequence for said first input molecule, and said NOT gate DNAzyme is deactivated; alternatively if only said first input molecule is present, said NOT gate DNAzyme is activated and cleaves said target sequence.

19. The library of claim 18, comprising an ANDNOT gate DNAzyme, said ANDNOT gate DNAzyme comprising said NOT gate DNAzyme and said AND gate DNAzyme, such that when both input molecules are present, said target sequence is cleaved.

20. The library of claim 19, comprising an XOR gate DNAzyme, said XOR gate DNAzyme comprising a plurality of ANDNOT gate DNAzymes.

21. The library of claim 18, comprising a NAND gate DNAzyme, said NAND gate DNAzyme comprising a plurality of NOT gate DNAzymes, such that said NAND gate DNAzyme is active unless all input molecules are present.

22. The library of claim 14, comprising an OR gate DNAzyme, said OR gate DNAzyme activating upon receiving either of two input molecules, said OR gate DNAzyme comprising two different YES gate DNAzymes joined such that once either input molecule binds, said OR gate DNAzyme is activated and cleaves said target sequence.


SEQUENCE LISTING (36 sequences)

SEQ ID NO: 1 - DNA, 34 bp, artificial
  DNAzyme
cgggaggaaggctagctacaacgagaggcgttgn

SEQ ID NO: 2 - RNA, 20 nt, artificial
  DNAzyme substrate
caacgccucguuccucccgn

SEQ ID NO: 3 - DNA, 27 bp, artificial
  computational DNA construct
cgggaggaaggctagctcatcttgcct

SEQ ID NO: 4 - DNA, 27 bp, artificial
  computational DNA construct
agctatgccagacaacgagaggcgttg

SEQ ID NO: 5 - DNA, 27 bp, artificial
  computational DNA construct
cgggaggaaggctacctcatcttgcct

SEQ ID NO: 6 - DNA, 28 bp, artificial
  computational DNA construct
cgggaggaaggctagctctgataagcta

SEQ ID NO: 7 - DNA, 27 bp, artificial
  computational DNA construct
tcaacatcagtacaacgagaggcgttg

SEQ ID NO: 8 - DNA, 28 bp, artificial
  computational DNA construct
cgggaggaaggctagctggtctcaggga

SEQ ID NO: 9 - DNA, 27 bp, artificial
  computational DNA construct
tcacaagttagacaacgagaggcgttg

SEQ ID NO: 10 - DNA, 71 bp, artificial
  computational DNA construct
cgggaggaaggctagctctgataagctatcacaagttagggtctcagggatagcttatcagagctagcctn

SEQ ID NO: 11 - DNA, 26 bp, artificial
  computational DNA construct
cgggaggaaggctagctcgtgatagg

SEQ ID NO: 12 - DNA, 25 bp, artificial
  computational DNA construct
aatgctaatacaacgagaggcgttg

SEQ ID NO: 13 - DNA, 23 bp, artificial
  computational DNA construct
acccctatcacgattagcattaa

SEQ ID NO: 14 - DNA, 25 bp, artificial
  computational DNA construct
ggcaagatgacaacgagaggcgttg

SEQ ID NO: 15 - DNA, 21 bp, artificial
  computational DNA construct
agctatgccagcatcttgcct

SEQ ID NO: 16 - DNA, 68 bp, artificial
  computational DNA construct
cgggaggaaggctagctctggcatagctcacaagttagggtctcagggagctatgccagagctagcct

SEQ ID NO: 17 - DNA, 25 bp, artificial
  computational DNA construct
gggaggaaggctagctctggcatag

SEQ ID NO: 18 - DNA, 24 bp, artificial
  computational DNA construct
ggcaagatgacaacgagaggcgtt

SEQ ID NO: 19 - DNA, 24 bp, artificial
  computational DNA construct
cgggaggaaggctagctctaactt

SEQ ID NO: 20 - DNA, 59 bp, artificial
  computational DNA construct
ttgtggtctcagggaatgccagcatcttgccttccctgagaccacaacgagaggcgttg

SEQ ID NO: 21 - DNA, 22 bp, artificial
  computational DNA construct
tcacaagttagggtctcaggga

SEQ ID NO: 22 - DNA, 68 bp, artificial
  computational DNA construct
cgggaggaaggctagctctggcatagctccctgagaccctaacttgtgagctatgccagagctagcct

SEQ ID NO: 23 - DNA, 66 bp, artificial
  computational DNA construct
tcgttgtactgatgttgaagctatgccagcatcttgccttcaacatcagtacaacgagaggcgttg

SEQ ID NO: 24 - DNA, 65 bp, artificial
  computational DNA construct
cgggaggaaggctagctctgataagctatcacaagttagggtctcatagcttatcagagctagcc

SEQ ID NO: 25 - DNA, 67 bp, artificial
  computational DNA construct
tcgttgtctaacttgtgatcaacatcagtctgataagctatcacaagttagacaacgagaggcgttg

SEQ ID NO: 26 - DNA, 69 bp, artificial
  computational DNA construct
cgggaggaaggctagctctgataagctaagctatgccagcatcttgccttagcttatcagagctagcct

SEQ ID NO: 27 - DNA, 62 bp, artificial
  computational DNA construct
tcgttgtctaacttgtgaatgccagcatcttgccttcacaagttagacaacgagaggcgttg

SEQ ID NO: 28 - DNA, 64 bp, artificial
  computational DNA construct
cgggaggaaggctagctggtctcagggaagctatgccagcatctttccctgagaccagctagcc

SEQ ID NO: 29 - RNA, 23 nt, artificial
  input to the computational DNA construct
uuaaugcuaaucgugauaggggu

SEQ ID NO: 30 - RNA, 21 nt, artificial
  input to computational DNA construct
aggcaagaugcuggcauagcu

SEQ ID NO: 31 - RNA, 22 nt, artificial
  input to computational DNA construct
ucccugagacccuaacuuguga

SEQ ID NO: 32 - RNA, 22 nt, artificial
  input to computational DNA construct
uagcuuaucagacugauguuga

SEQ ID NO: 33 - DNA, 20 bp, artificial
  fluorophore substrate
caangcctcgutcctcccgn

SEQ ID NO: 34 - DNA, 38 bp, human
taatacgactcactatagttcgggtagtggaaaaccag

SEQ ID NO: 35 - DNA, 20 bp, human
tttccgcaacaagtcctctt

SEQ ID NO: 36 - DNA, 19 bp, artificial
  fluorophore substrate
caangcctcgutcctcccn